(19) United States
(12) Patent Application Publication
(10) Pub. No.: US 2024/0296951 A1
(43) Pub. Date: Sep. 5, 2024

(54) A COMPUTER IMPLEMENTED METHOD FOR DETERMINING A PROBABILITY OF A DISEASE IN AT LEAST ONE IMAGE REPRESENTATIVE OF AN EYE

(71) Applicant: Oncotech Ltd, London (GB)

(72) Inventors: Marat Kalimulov, Ekaterinburg (RU); Anastasiia Rusakova, Ekaterinburg (RU); Rustem Amirov, Ufa (RU); Andre Rafnsson, Vejbystrand (SE); Shamil Gantsev, Ufa (RU)

(73) Assignee: Oncotech Ltd, London (GB)

(21) Appl. No.: 18/578,383

(22) PCT Filed: Jul. 7, 2022
(86) PCT No.: PCT/EP2022/068940

§ 371 Date: Jan. 11, 2024

(30) Foreign Application Priority Data
Jul. 14, 2021 (SE) 2150942-7

(51) Int. Cl. G16H 50/20; G06T 7/00; G16H 50/30

(52) U.S. Cl. CPC G16H 50/20; G06T 7/0012; G16H 50/30; G06T 2207/30041

(57) ABSTRACT

The present disclosure relates to a computer implemented method (400) for determining a probability of a disease in at least one image representative of an eye. The method (400) comprising: identifying (S102) two or more eye features in the at least one image, for each identified feature, identifying (S104) at least one element associated with the identified feature, for the at least one element of each identified feature, determining (S106) a quantitative value indicative of the at least one elements significance for the disease, determining (S108) a position of the at least one element of each identified feature, determining (S110) the probability of the disease based on the quantitative values and position of the at least one element for each identified feature.

### TECHNICAL FIELD

The present invention relates to risk assessment of diseases. More particularly, it relates to a method for determining a probability of a disease in at least one image representative of an eye.

### BACKGROUND OF THE INVENTION

The application of new technologies within the MedTech industry has seen great improvements during the last couple of years. More and more advanced technologies are now available for doctors, for example when conducting medical examination.

Different types of technologies are today used when diagnosing or trying to predict the medical state of a person. One technology is imaging and especially imaging of an iris. The use of the iris to determine a person's medical state is today based on for example iridology maps.

However, there are improvements to be made within this field of diagnosing the medical state of the person. At least in regard to the importance of early diagnosing diseases. Thus, there is a need for a simpler and more efficient method of diagnosing based on imaging of the eye.

### SUMMARY OF THE INVENTION

In view of the above, it is at least partly an object of the present invention to provide a method for determining a probability of a disease in at least one image representative of an eye.

It has been realized that by assessing multiple features and elements of a mammal eye, a probability of the occurrence of a disease can be determined in an accurate and effective way. The method is further advantageous in that it required no contact with the subject.

Throughout this disclosure the eye refers not only to the human eye itself, but it should be understood to also cover adjacent areas to the eye. In other words for example the contents of the orbit such as the iris, pupil, protein, lacrimal duct, eye vessels, eyelid, skin under the eye.

According to a first aspect, a computer implemented method for determining a probability of a disease in at least one image representative of an eye is provided. The method comprising: identifying two or more eye features in the at least one image, for each identified feature, identifying at least one element associated with the identified feature, for the at least one element of each identified feature, determining a quantitative value indicative of the at least one elements significance for the disease, determining a position of the at least one element of each identified feature, and determining the probability of the disease based on the quantitative values and position of the at least one element for each identified feature.

The quantitative values may be determined by a machine learning model.

Determining the probability of the disease may comprise determining a weighted sum of the quantitative values of the at least one element of each identified feature, wherein each weight relates to a significance that the corresponding element has when assessing an occurrence of the disease. The probability of the disease may be determined by comparing the weighted sum with a set of predetermined sums with known probabilities of the disease and obtaining the probability of the disease as the known probability of the predetermined sum that best matches the weighted sum.

The elements associated with the eye features may be interpreted as being a sub-element of the eye features. In other words, one eye feature may comprise one or more elements. Put differently, the eye features may be seen as eye visuals of a first level, and the elements as eye visuals of a second level. Looking at signs of the disease in eye visuals in two levels may be advantageous in that it gives a more accurate result while also keeping the computational costs low. Further, looking at multiple features and elements makes it possible to reduce a number of false positives and false negatives among the results.

The position of the at least one element may be the position of the element in the eye. In other words, the position of the element in relation to other parts of the eye. Determining the position of the at least one element may be advantageous in that it can provide more information about the probability of having the disease. For example, if a certain element is identified in the iris of the eye may be more or less indicative of the disease than if the element was found in the white of the eye.

The at least one image representative of the eye may be multiple images of the same eye. Alternatively, at least one image of each eye of a subject may be used. An advantage of using at least one image of each eye may be that signs of the disease can be more or less prominent in either eye, or even only existing in one of the eyes. Thus, a more efficient and accurate assessment can be performed and the risk of missing the disease is reduced.

The eye features may for example be an iris, a pupil, a white of the eye, external organs of the lacrimation, eyelids, and eye area such as skin around the eye.

The elements may for example be pupillary belt, pupillary boarder, autonomous ring, cylinder belt, adaptation arcs, dystrophic rim, lymphatic rose garden, nartia ring, local elements such as blackheads, lacunae, pigment and toxic spots, toxic sector, areas with different colors, iris color, iris shape, condition of pupil, location of pupil, blood vessel density, hemorrhage, blotches, lacrimal stream, lacrimal lake, lacrimal meat, lacrimal papillae with lacrimal dots, skin formations, skin inflammation, swelling, drooping eyelids, skin and nevus.

The step of determining the probability of the disease may comprise determining a confidence score between the identified elements and a library of elements associated with a known probability of the disease. The method may further comprise, identifying the disease as present in the eye, if the confidence score is above a threshold. In other words, if the confidence score is above the threshold, the disease can be identified with confidence.

The quantitative value may be a numerical value quantifying the identified element. The quantitative value may be descriptive of one or more parameters of the identified element.

The method may further comprise: determining a weighted sum of the quantitative values of the at least one element of each identified feature, wherein each weight relates to a significance that the corresponding element has when assessing a grade of the disease, comparing the weighted sum with a set of predetermined sums with known grades of the disease, and obtaining the grade of the disease as the known grade of the predetermined sum that best matches the weighted sum.

The term “grade of the disease” may be interpreted as a severity of the disease. Alternatively, it may be interpreted as a stage or degree of the disease. In other words, how far progressed the disease are.

A possible associated advantage of the method as described above is that the development of the disease can be monitored to, for example, see if there are cause for starting treatment or to see if a current treatment is working. In other words, the progress of an identified disease can be monitored. The monitoring of the disease can be done in a cost and time effective way since it does not require the subject to visit a hospital.

Further, an advantage of weighting the elements may be that different element may be more indicative of the disease than others. Thus, a more accurate results may be achieved.

The act of determining the probability of the disease may be based on a dataset of images representative of eyes with or without the disease. The dataset may comprise of multiple subsets of images. Each subset of images may be related to a specific disease.

The act of determining the probability of the disease may be performed by a machine learning, ML, model, such as a neural network. The ML model may be trained using the dataset of images representative of eyes with or without the disease.

The neural network may be a combined convolutional and recurrent neural network.

The act of identifying the two or more eye features in the at least one image and/or identifying the at least one element associated with the identified feature for each feature may be performed by an ML model, such as a neural network. The ML model may be trained using a dataset of images representative of eyes with or without the disease where the features and/or elements has already been identified.

The neural network may be a combined convolutional and recurrent neural network.

The quantitative value of the elements may be based on at least one of a thickness, type, shape, size, location, presence or non-presence, bulging or retraction, clarity, visible gaps, breaks, normal or swelling, inflammation, deformation, quantity, brightness, color, and shade of the elements. It is hereby noted that the quantitative value of one identified element may be based on different parameters than another identified element.

The method may further comprise receiving a user input indicating what disease to look for and selecting only the identified features that are relevant for the disease. Relevant for the disease may be that they are indicative of the occurrence of the disease.

A possible associated advantage may be that computational resources may be saved, and less data transferred by knowing what features and elements is to be analyzed.

According to a second aspect, a user device configured for determining a probability of a disease in at least one image representative of an eye, the user device comprising, a camera configured to capture the at least one image, and circuitry configured to execute: an identifying function configured to; identify two or more eye features in the at least one image, and for each identified feature, identify at least one element associated with the identified feature, a determining function configured to determine, for the at least one element of each identified feature, a quantitative value indicative of the at least one elements significance for the disease, a positioning function configured to determine a position of the at least one element of each identified feature, and a probability function configured to determine the probability of the disease based on the quantitative values and position of the at least one element for each identified feature.

According to a third aspect, server configured for determining a probability of a disease in at least one image representative of an eye is provided. The server comprises circuitry configured to execute: an identifying function configured to; identify two or more eye features in the at least one image, and for each identified feature, identify at least one element associated with the identified feature, a determining function configured to determine, for the at least one element of each identified feature, a quantitative value indicative of the at least one elements significance for the disease, a positioning function configured to determine a position of the at least one element of each identified feature, and a probability function configured to determine the probability of the disease based on the quantitative values and position of the at least one element for each identified feature.

The circuitry may be further configured to execute: a weighting function configured to determine a weighted sum of the quantitative values of the at least one element of each identified feature, wherein each weight relates to a significance that the corresponding element has when assessing the grade of the disease, a comparing function configured to compare the weighed sum with a set of predetermined sums of known grades of the disease, and an obtaining function configured to obtain a grade of the disease as the known grade of the predetermined sum that best matches the weighted sum.

The circuitry may be further configured to execute a receiving function configured to receive a user input indicating what disease to look for, and wherein the identifying function identifies elements only for features relevant for the indicated disease.

According to a fourth aspect, a system for determining a probability of a disease in at least one image representative of an eye is provided. The system comprising: the server according to the third aspect, a first database of images representative of eyes with or without the disease, and a second database of statistical weights, wherein each weight relates to a significance of an element when determining a probability of the disease.

A possible associated advantage is that a low-cost system is achieved. It requires no expensive medical equipment or disposable material.

The first database may comprise images representative of eyes with different diseases. Put differently, the first database may comprise a subset of images of eyes with one disease, and another subset of images of eyes with another disease.

The second database may comprise statistical weights for one or more different diseases.

The system may further comprise a third database of statistical weights wherein each weight relates to a significance of an element when determining a grade of the disease. The third database may comprise statistical weights for one or more different diseases.

The system may further comprise a user device, wherein the user device comprises a camera configured to capture the at least one image of the eye of the user.

As non-limiting examples, the user device may be a smart phone, a tablet, a computer with an internal or external camera or the like.

The above-mentioned features and advantages of the first aspect, when applicable, apply to the second, third and fourth aspects as well. To avoid undue repetition, reference is made to the above.

Still other objectives, features, aspects, and advantages of the invention will appear from the following detailed description as well as from the drawings. The same features and advantages described with respect to one aspect are applicable to the other aspects unless explicitly stated otherwise.

### BRIEF DESCRIPTION OF THE DRAWINGS

The above and other aspects of the present inventive concept will now be described in more detail, with reference to appended drawings showing variants of the invention. The figures should not be considered limiting the invention to the specific variant; instead they are used for explaining and understanding the inventive concept.

As illustrated in the figures, the sizes of layers and regions are exaggerated for illustrative purposes and, thus, are provided to illustrate the general structures of variants of the present inventive concept. Like reference numerals refer to like elements throughout.

FIG. 1A is a flow chart illustrating the steps of a computer implemented method for determining a probability of a disease in at least one image representative of an eye.

FIG. 1B is a flow chart illustrating the steps of another computer implemented method for determining a probability of a disease in at least one image representative of an eye.

FIG. 2 schematically illustrates, by way of example, a server configured for determining a probability of a disease in at least one image representative of an eye.

FIG. 3 schematically illustrates, by way of example, a system for determining a probability of a disease in at least one image representative of an eye.

FIG. 4 schematically illustrates, by way of example, a user device configured for determining a probability of a disease in at least one image representative of an eye.

### DETAILED DESCRIPTION

The present inventive concept will now be described more fully hereinafter with reference to the accompanying drawings, in which currently preferred variants of the inventive concept are shown. This inventive concept may, however, be implemented in many different forms and should not be construed as limited to the variants set forth herein; rather, these variants are provided for thoroughness and completeness, and fully convey the scope of the present inventive concept to the skilled person.

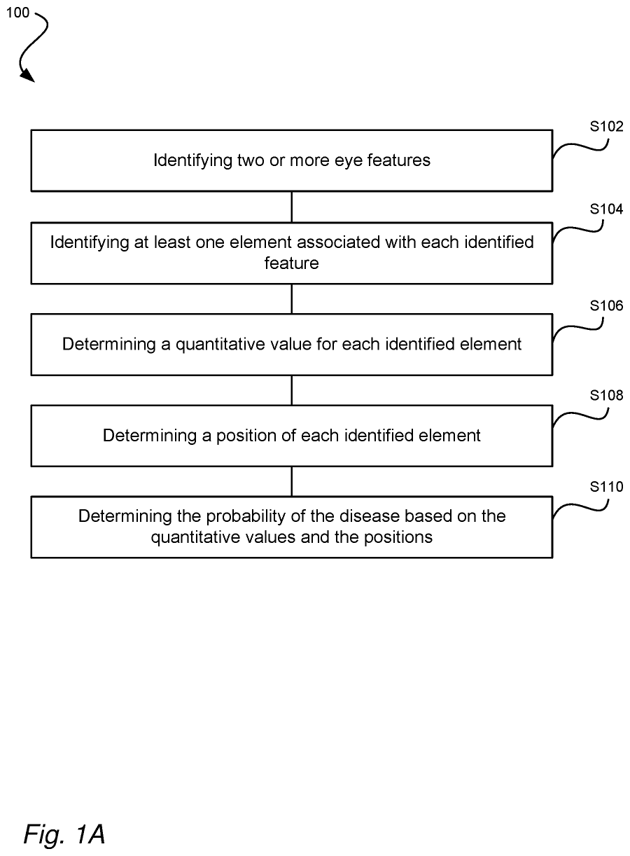

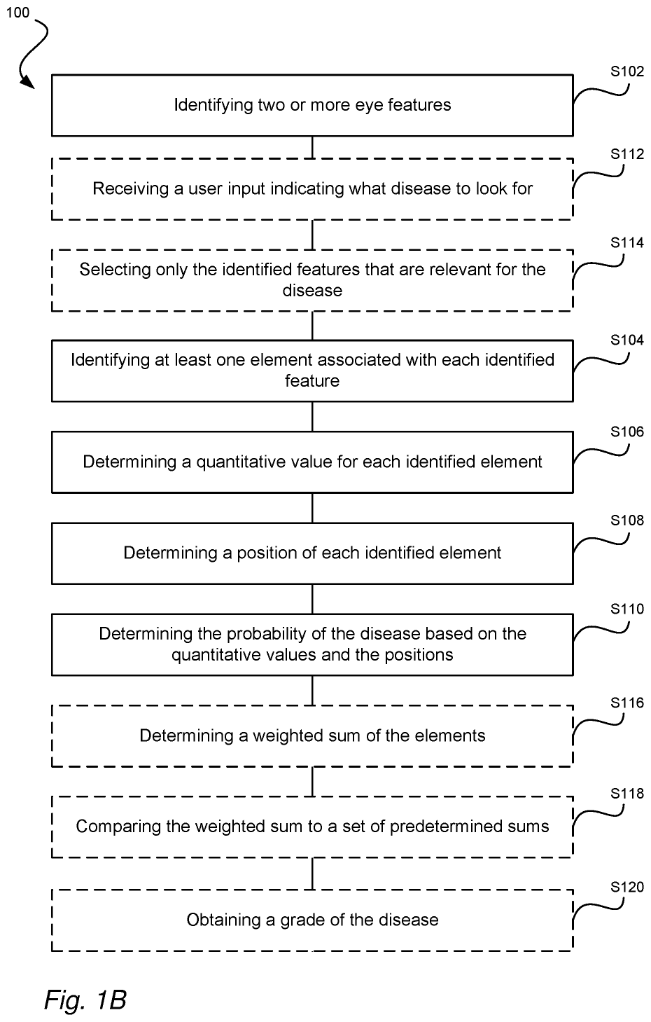

A computer implemented method for determining a probability of a disease in at least one image representative of an eye, as well as a user device, a server, and a system thereof, will now be described with reference to FIG. 1 to FIG. 4.

FIG. 1A is a flow chart illustrating the steps of the method 100 for determining a probability of a disease in at least one image representative of an eye. Below, the different steps is described in more detail. Even though illustrated in a specific order, the steps of the method 100 may be performed in any suitable order, in parallel, as well as multiple times.

Two or more eye features are identified S102 in the at least one image. The act of identifying S102 the eye features may be performed by a machine learning model. The machine learning model may comprise a neural network.

For each identified eye feature, at least one element associated with the identified feature is identified S104. The act of identifying S104 the elements may be performed by a machine learning model. The machine learning model may comprise a neural network. Thus, signs of the disease can be found by looking at two levels of feature details.

For the at least one identified element of each identified feature, a quantitative value indicative of the at least one elements significance for the disease is determined S106. The quantitative value of the elements may be based on at least one of a thickness, type, shape, size, location, presence or non-presence, bulging or retraction, clarity, visible gaps, breaks, normal or swelling, inflammation, deformation, quantity, brightness, color, and shade of the elements. The quantitative value can be formed on an empirical scale with a starting point of a healthy state with value 0 or 1 and an ending point at a high risk of disease with value of for instance 10, 100 or 500. The quantitative value can be further formed on other intermediate definitions and arbitrary values. The quantitative values can be determined based on grouping of a control group of patients according to the grade of the disease. Alternatively, or in combination, the quantitative values can be determined based on grouping of signs of a given control group of patients by quantitative values (e.g. size, color intensity etc.). Alternatively, or in combination, the quantitative values can be determined based on allocation of those signs that quantitatively correlate with the grade of the disease in the control group of patients. Alternatively, or in combination, the quantitative values can be determined based on determination of the grade of the disease to the empirical value of the scale.

A position of the at least one identified element of each identified feature is determined S108.

The probability of the disease is determined S110, based on the quantitative value and position of the at least one element for each identified feature. Determining S110 the probability of the disease may be further based on a dataset of images representative of eyes with or without the disease. The identified elements and/or features may be compared with elements and/or features of the images representative of eyes with or without the disease in the dataset. Determining S110 the probability of the disease may be performed by a machine learning model. The machine learning model may be a neural network.

FIG. 1B illustrates another example of the method 100 as discussed above in connection with FIG. 1A. The method 100 may further comprise the following steps.

Optionally, a user input is received S112 indicating what disease to look for.

Optionally, only the identified features that are relevant for the indicated disease is selected S114. Thus, when identifying S104 at least one element associated with each identified feature, only the selected features may need to be identified. Correspondingly, quantitative values and positions may only need to be determined for the selected features.

Optionally, a weighted sum of the quantitative values of the at least one element of each identified feature is determined S116. Each weight may relate to a significance that the corresponding element has when assessing a grade of the disease.

Optionally, the weighted sum is compared S118 with a set of predetermined sums of known grades of the disease.

Optionally, a grade of the disease is obtained S120 as the known grade of the predetermined sum that best matches the weighted sum. Thus, the grade of the disease can be determined by comparing the weighted sum to the set of predetermined sums of known grades of the disease.

As with the method 100 described above in connection with FIG. 1a, the additional optional steps described in connection with FIG. 1b may be performed in any suitable order, in parallel, as well as multiple times.

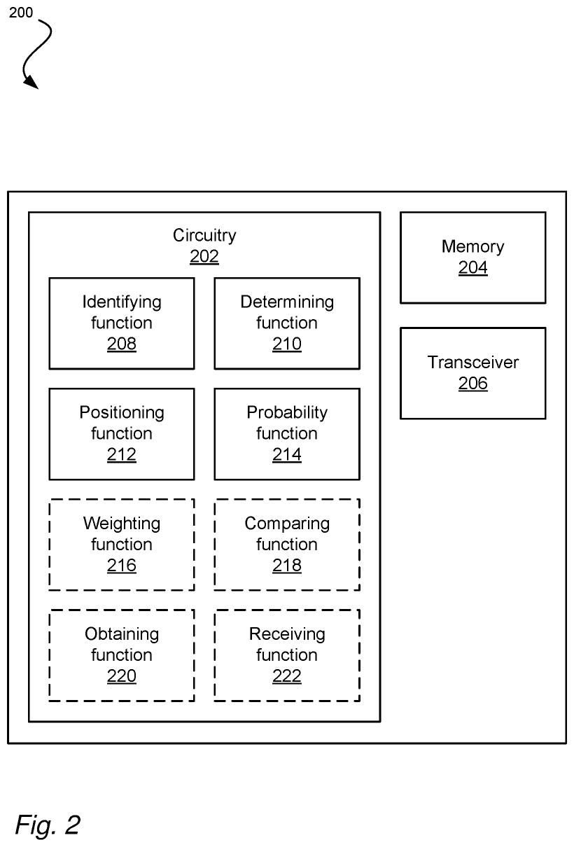

FIG. 2 illustrates a server 200 configured for determining a probability of a disease in at least one image representative of an eye. The server 200 comprises circuitry 102.

The circuitry 202 may be configured to carry out overall control of functions and operations of the server 200. The circuitry 202 may include a processor, such as a central processing unit (CPU), microcontroller, or microprocessor. The processor may be configured to execute program code stored in a memory 204 of the server 200, in order to carry out functions and operations of the server 200. The memory 204 may be one or more of a buffer, a flash memory, a hard drive, a removable media, a volatile memory, a non-volatile memory, a random access memory (RAM), or another suitable device. In a typical arrangement, the memory 204 may include a non-volatile memory for long term data storage and a volatile memory that functions as server memory for the circuitry 202. The memory 204 may exchange data with the processor over a data bus. Accompanying control lines and an address bus between the memory 204 and the processor also may be present. Functions and operations of the circuitry 202 may be embodied in the form of executable logic routines (e.g., lines of code, software programs, etc.) that are stored on a non-transitory computer readable recording medium (e.g., the memory) of the server 200 and are executed by the processor. Furthermore, the functions and operations of the server 200 may be a stand-alone software application or form a part of a software application that carries out additional tasks related to the server 200. The described functions and operations may be considered a method that the corresponding device is configured to carry out. Such as the method 100 discussed above in connection with FIGS. 1A and 1B. Also, while the described functions and operations may be implemented in software, such functionality may as well be carried out via dedicated hardware or firmware, or some combination of hardware, firmware and/or software.

The server 200 may further comprise a transceiver 206 configured to enable the server 200 to communicate with other devices. The other devices may for example be other servers, external databases, or a user device.

The circuitry 202 is configured to execute an identification function 208. The identification function 208 may be configured to identify two or more eye features in the at least one image. The identification function 208 may be further configured to, for each identified feature, identify at least one element associated with the identified feature. The identification function 208 may use a machine learning model to identify the eye features and/or elements.

The circuitry 202 is further configured to execute a determining function 210. The determining function 210 may be configured to determine, for the at least one element of each identified feature, a quantitative value indicative of the at least one elements significance for the disease. Put differently, the determining function 210 may be configured to determine a quantitative value for every identified element.

The circuitry 202 is further configured to execute a positioning function 212. The positioning function 212 may be configured to determine a position of the at least one element of each identified feature. Put differently, the positioning function 212 may be configured to determine a position of every identified element.

The circuitry 202 is further configured to execute a probability function 214. The probability function 214 may be configured to determine the probability of the disease. The probability of the disease may be based on the quantitative values and/or position of the at least one element for each identified feature. The probability may be determined by using a machine learning model.

The circuitry 202 may be further configured to execute a weighting function 216. The weighting function 216 may be configured to determine a weighted sum of the quantitative values of the at least one element of each identified feature. Each weight may relate to a significance that the corresponding element has when assessing the grade of the disease.

The circuitry 202 may be further configured to execute a comparing function 218. The comparing function 218 may be configured to compare the weighed sum with a set of predetermined sums of known grades of the disease.

The circuitry 202 may be further configured to execute an obtaining function 220. The obtaining function 220 may be configured to obtain the grade of the disease as the known grade of the predetermined sum that best matches the weighted sum.

The circuitry 202 may be further configured to execute a receiving function 222. The receiving function 222 may be configured to receive a user input indicating what disease to look for. Upon receiving the user input indicating the disease, the identifying function 208 may only identify elements for eye features relevant for the indicated disease.

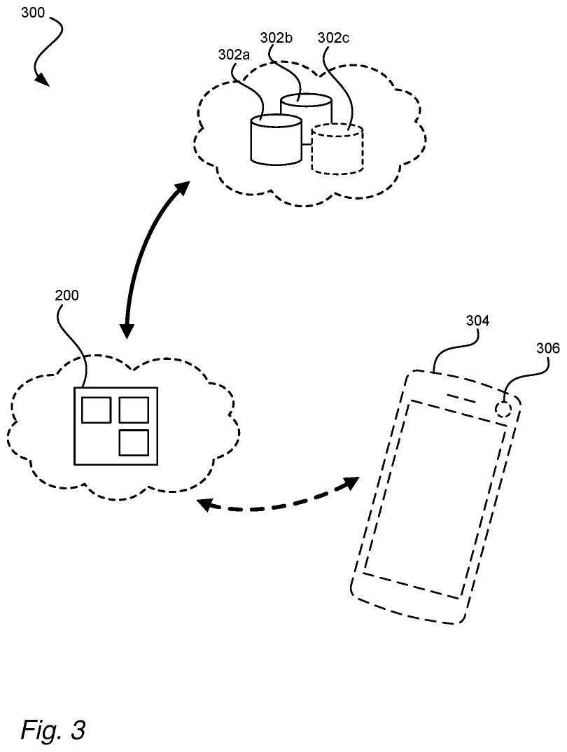

FIG. 3 illustrates, by way of example, a system 300 for determining a probability of a disease in at least one image representative of an eye.

The system 300 comprises the server 200 as described above in connection with FIG. 2. The server 200 may be provided as a cloud service.

The system 300 further comprises a first database 302a. The first database 302a may comprise images representative of eyes with or without a disease. The first database 302a may comprise one or more subsets, wherein each subset is related to one type of disease.

The system 300 further comprises a second database 302b. The second database 302b may comprise statistical weights. Each weight of the second database 302b may relate to a significance of an element when determining a probability of the disease. The second database 302b may comprise one or more subsets, wherein each subset is related to one type of disease.

The system 300 may further comprise a third database 302c. The third database 302c may comprise statistical weights. Each weight of the third database 302c may relate to a significance of an element when determining a grade of a disease. The third database 302c may comprise one or more subsets, wherein each subset is related to one type of disease.

The databases of the systems may be provided as external databases to the server 200. For example, the databases may be provided as a cloud service as herein illustrated. Thus, the server may be communicatively connected to the databases 302a-c. Alternatively, the databases may be provided as internal databases to the server 200.

The system 300 may further comprise a user device 304. The user device 304 may be communicatively connected to the server 200. The user device may comprise a camera 306. The camera 306 may be configured to capture the at least one image of the eye of the user. The user device may be a smart phone, a tablet, or the like.

In one exemplary embodiment, the user captures one or more images of its left, right or both left and right eyes with the user device 304. The user device 304 may then transmit the one or more images to the server 200. The server 200 may perform preprocessing of the one or more images. The server 200 may then determine the probability of a disease based on the one or more images of the eyes. The server may further determine a grade of the identified disease.

In addition to transmitting the one or more images, the user device 304 may transmit an indication to what disease to look for in the one or more images. Upon receiving the indication of what disease to look for, the server may extract only information pertaining to the indicated disease from the databases 302a-302c. Thus, less data has to be transmitted and computational resources can be saved.

The determined probability and/or grade of the disease may be transmitted from the server to the user device. These results may then be used in consultancy with a doctor for completing a diagnosis.

The thick arrows should be interpreted as ways of communication. The dashed lines and objects should be interpreted as optionally being part of the system.

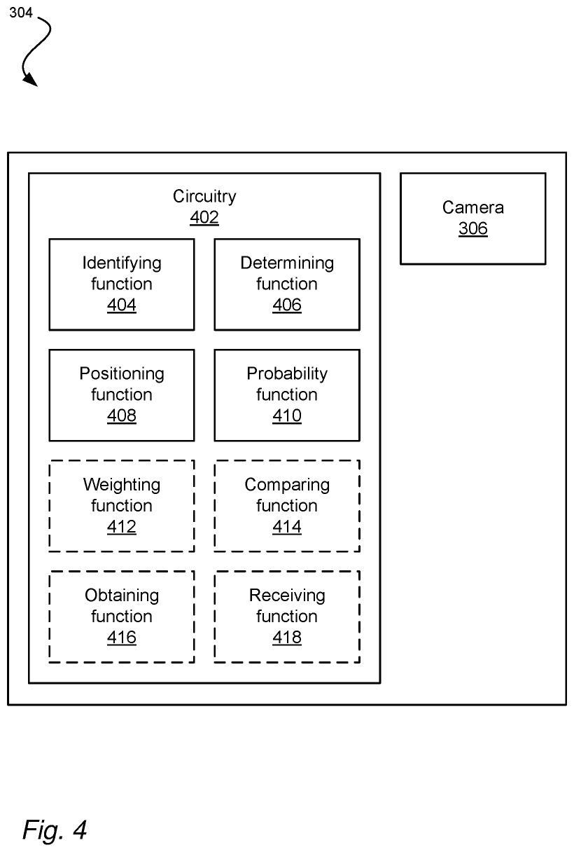

FIG. 4 schematically illustrates the user device 304, or client, as discussed above in connection with FIG. 3. The user device may be configured for determining a probability of a disease in at least one image representative of an eye.

The user device 304 comprises a camera configured to capture the at least one image representative of an eye.

The user device 304 further comprises circuitry 402.

The circuitry 402 may be configured to carry out overall control of functions and operations of the user device 304. The circuitry 402 may include a processor, such as a central processing unit (CPU), microcontroller, or microprocessor. The processor may be configured to execute program code stored in a memory of the user device 304, in order to carry out functions and operations of the user device 304. The memory may be one or more of a buffer, a flash memory, a hard drive, a removable media, a volatile memory, a non-volatile memory, a random access memory (RAM), or another suitable device. In a typical arrangement, the memory may include a non-volatile memory for long term data storage and a volatile memory that functions as server memory for the circuitry 402. The memory may exchange data with the processor over a data bus. Accompanying control lines and an address bus between the memory and the processor also may be present. Functions and operations of the circuitry 402 may be embodied in the form of executable logic routines (e.g., lines of code, software programs, etc.) that are stored on a non-transitory computer readable recording medium (e.g., the memory) of the user device 304 and are executed by the processor. Furthermore, the functions and operations of the user device 304 may be a stand-alone software application or form a part of a software application that carries out additional tasks related to the user device 304. The described functions and operations may be considered a method that the corresponding device is configured to carry out. Such as the method 100 discussed above in connection with FIGS. 1A and 1B. Also, while the described functions and operations may be implemented in software, such functionality may as well be carried out via dedicated hardware or firmware, or some combination of hardware, firmware and/or software.

The user device 304 may further comprise a transceiver configured to enable the user device 304 to communicate with other devices. The other devices may for example be a server, external databases, or other user devices.

The circuitry 402 is configured to execute an identification function 404. The identification function 404 may be configured to identify two or more eye features in the at least one image. The identification function 404 may be further configured to, for each identified feature, identify at least one element associated with the identified feature. The identification function 404 may use a machine learning model to identify the eye features and/or elements.

The circuitry 402 is further configured to execute a determining function 406. The determining function 406 may be configured to determine, for the at least one element of each identified feature, a quantitative value indicative of the at least one elements significance for the disease. Put differently, the determining function 406 may be configured to determine a quantitative value for every identified element.

The circuitry 402 is further configured to execute a positioning function 408. The positioning function 408 may be configured to determine a position of the at least one element of each identified feature. Put differently, the positioning function 408 may be configured to determine a position of every identified element.

The circuitry 402 is further configured to execute a probability function 410. The probability function 410 may be configured to determine the probability of the disease. The probability of the disease may be based on the quantitative values and/or position of the at least one element for each identified feature. The probability may be determined by using a machine learning model.

The circuitry 402 may be further configured to execute a weighting function 412. The weighting function 412 may be configured to determine a weighted sum of the quantitative values of the at least one element of each identified feature. Each weight may relate to a significance that the corresponding element has when assessing the grade of the disease.

The circuitry 402 may be further configured to execute a comparing function 414. The comparing function 414 may be configured to compare the weighed sum with a set of predetermined sums of known grades of the disease.

The circuitry 402 may be further configured to execute an obtaining function 416. The obtaining function 416 may be configured to obtain the grade of the disease as the known grade of the predetermined sum that best matches the weighted sum.

The circuitry 418 may be further configured to execute a receiving function 418. The receiving function 418 may be configured to receive a user input indicating what disease to look for. Upon receiving the user input indicating the disease, the identifying function 404 may only identify elements for eye features relevant for the indicated disease.

It should be noted that the steps of the method 100 as described above in connection with FIGS. 1A and 1B may all be performed by the server 200 as described in connection with FIG. 2. Alternatively, all steps of the method 100 may be performed by the user device 304 as described in connection with FIG. 4. Alternatively, a part of the method 100 may be performed by the server 200 while another part of the method 100 may be performed by the user device. Further, the functions of the server 200 may be distributed over multiple servers. Similarly, the functions of the user device 304 may be distributed over multiple user devices.

Generally, in the context of this application the term “circuitry, processor or computer” refers to any electronic device comprising a processor, such as a general-purpose central processing unit (CPU), a specific purpose processor such as field-programmable gate array or a microcontroller. A processor, computer or circuitry is capable of receiving data (an input), of performing a sequence of predetermined operations thereupon, and of producing thereby a result in the form of information or signals (an output). Depending on context, the term “computer” will mean either a processor in particular or can refer more generally to a processor in association with an assemblage of interrelated elements contained within a single case or housing.

The systems and methods described herein may be embodied by a computer program or a plurality of computer programs, which may exist in a variety of forms both active and inactive in a single computer system or across multiple computer systems. For example, they may exist as software program(s) comprised of program instructions in source code, object code, executable code, or other formats for performing some of the steps. Any of the above may be embodied on a computer readable medium, which include storage devices and signals, in compressed or uncompressed form.

A non-transitory computer-readable storage medium having stored thereon instructions for implementing the method 100 as described above in connection with FIGS. 1A and 1B, when executed on a device having processing capabilities is also provided.

Additionally, variations to the disclosed variants can be understood and effected by the skilled person in practicing the claimed invention, from a study of the drawings, the disclosure, and the appended claims.

1. A computer implemented method for determining a probability of a disease in at least one image representative of an eye, the method comprising:
identifying two or more eye features in the at least one image,
for each identified feature, identifying at least one element associated with the identified feature,
for the at least one element of each identified feature, determining a quantitative value indicative of the at least one elements significance for the disease,
determining a position of the at least one element of each identified feature,
determining the probability of the disease based on the quantitative values and position of the at least one element for each identified feature.

2. The method according to claim 1, further comprising:
determining a weighted sum of the quantitative values of the at least one element of each identified feature, wherein each weight relates to a significance that the corresponding element has when assessing a grade of the disease,
comparing the weighted sum with a set of predetermined sums of known grades of the disease, and
obtaining a grade of the disease as the known grade of the predetermined sum that best matches the weighted sum.

3. The method according to claim 1, wherein determining the probability of the disease is further based on a dataset of images representative of eyes with or without the disease.

4. The method according to claim 1, wherein determining the probability of the disease is performed by a machine learning model, such as a neural network.

5. The method according to claim 1, wherein identifying the two or more eye features in the at least one image and/or identifying the at least one element associated with the identified feature for each feature is performed by a machine learning model, such as a neural network.

6. The method according to claim 1, wherein the quantitative value of the elements is based on at least one of a thickness, type, shape, size, location, presence or non-presence, bulging or retraction, clarity, visible gaps, breaks, normal or swelling, inflammation, deformation, quantity, brightness, color, and shade of the elements.

7. The method according to claim 1, further comprising:
receiving a user input indicating what disease to look for,
selecting only the identified features that are relevant for the indicated disease.

8. A user device configured for determining a probability of a disease in at least one image representative of an eye, the user device comprising:
a camera configured to capture the at least one image, and circuitry configured to execute:
an identifying function configured to:
identify two or more eye features in the at least one image, and
for each identified feature, identify at least one element associated with the identified feature,
a determining function configured to determine, for the at least one element of each identified feature, a quantitative value indicative of the at least one elements significance for the disease,
a positioning function configured to determine a position of the at least one element of each identified feature, and
a probability function configured to determine the probability of the disease based on the quantitative values and position of the at least one element for each identified feature.

9. A server configured for determining a probability of a disease in at least one image representative of an eye, the server comprising circuitry configured to execute:
an identifying function configured to:
identify two or more eye features in the at least one image, and for each identified feature, identify at least one element associated with the identified feature,
a determining function configured to determine, for the at least one element of each identified feature, a quantitative value indicative of the at least one elements significance for the disease,
a positioning function configured to determine a position of the at least one element of each identified feature, and
a probability function configured to determine the probability of the disease based on the quantitative values and position of the at least one element for each identified feature.

10. The server according to claim 9, wherein the circuitry is further configured to execute:
a weighting function configured to determine a weighted sum of the quantitative values of the at least one element of each identified feature,
wherein each weight relates to a significance that the corresponding element has when assessing the grade of the disease,
a comparing function configured to compare the weighed sum with a set of predetermined sums of known grades of the disease, and
an obtaining function configured to obtain a grade of the disease as the known grade of the predetermined sum that best matches the weighted sum.

11. The server according to claim 9, wherein the circuitry is further configured to execute:
a receiving function configured to receive a user input indicating what disease to look for, and
wherein the identifying function identifies elements only for features relevant for the indicated disease.

12. A system for determining a probability of a disease in at least one image representative of an eye, the system comprising:
the server according to claim 9,
a first database of images representative of eyes with or without the disease, and,
a second database of statistical weights, wherein each weight relates to a significance of an element when determining a probability of the disease.

13. The system according to claim 12, wherein the system further comprises a third database of statistical weights wherein each weight relates to a significance of an element when determining a grade of the disease.

14. The system according to claim 12, wherein the system further comprises a user device, wherein the user device comprises a camera configured to capture the at least one image of the eye of the user.
